(19) United States
(12) Patent Application Publication
(10) Pub. No.: US 2011/0132359 A1
(43) Pub. Date: Jun. 9, 2011

(54) COMPACTABLE INHALING DEVICE

(76) Inventor: Thierry Poree, Saint-Pierre-Eglise (FR)

(21) Appl. No.: 13/058,427

(22) PCT Filed: Aug. 10, 2009
(86) PCT No.: PCT/EP2009/060321

§ 371 Date: Feb. 10, 2011

(30) Foreign Application Priority Data
Aug. 12, 2008 (FR) 0855527
Dec. 22, 2008 (FR) 0858925
Jul. 22, 2009 (FR) 0955115

(51) Int. Cl. A61M 15/00

(52) U.S. Cl. 128/203.21; 128/203.12; 128/203.29

(57) ABSTRACT

The invention relates to an inhaling device, especially for administration of inhaled treatments for management of bronchopulmonary disorders, comprising a substantially tubular, axial reservoir that delimits an inhaling chamber, of which a first end is joined to a dosing aerosol containing an active principle, and of which a second, opposite end is joined to a means for delivering the treatment, especially a mouthpiece or a face mask, and the reservoir comprises at least a first wall that can move between a compact position and a deployed position, in such a way as to allow said reservoir to adopt a first position, in which it occupies a reduced volume for transport thereof, and a second position, in which it is deployed in a substantially rigid form for use thereof.

The invention concerns an inhaling device for administration of inhaled treatments intended to treat bronchopulmonary disorders.

The invention concerns more particularly an inhaling device in particular for the administration of inhaled treatment intended to treat bronchopulmonary disorders, comprising a substantially tubular axial reservoir delimiting an inhalation chamber, a first end of which is joined to a dosing aerosol containing an active principle, and a second opposite end of which is attached to a treatment distribution means, in particular a mouth piece or a facial mask.

Numerous anti-asthmatic drugs intended to treat pathologies such as bronchopulmonary disorders, asthma, bronchopathies, or bronchiolitis are administered using dosing aerosols.

This design, well known from the prior art, is based on the principle of aerosol capsules. It consists of vaporising via an aerosol a specific dose of an active principle and inhaling this dose by inspiration.

Use of the dosing aerosol alone therefore requires good synchronisation between the action of the aerosol and the inhalation of the patient.

Therefore use of the dosing aerosol alone is not properly managed by many patients, in particular children.

In fact when the dosing aerosol is poorly used or when there is no synchronisation between ejection of the active principle and inhalation, too low a quantity of product reaches the lungs and the efficacy of the treatment is diminished or even eliminated.

To remedy this drawback, it is proposed to add inhalation chambers to the dosing aerosols known from the prior art in order to facilitate inhalation of the drug.

An inhalation chamber as known from the prior art usually comprises a reservoir placed between the dosing aerosol which is connected to one side and a tube, in particular terminating in a valve, fixed to the other end, on which can be attached a distribution means such as a rigid or flexible mouth piece, for single or repeated use, or a facial mask, in particular for young children.

The active principle is despatched into the chamber by pressure on the dosing aerosol, distributed into the reservoir and then inhaled by the patient who breathes calmly into the mouth piece or mask through his mouth.

This design avoids handling errors due to poor coordination between the action of the aerosol and the inhalation of the patient. It is often essential to treat asthma in an infants or bronchiolitis affecting young children in winter. Associated with a paediatric mask, the inhalation chamber constitutes the most practical means of administering aerosols to young patients.

However this design is restrictive, as an inhalation chamber coupled to an aerosol is more cumbersome than a dosing aerosol alone.

Inhalation chambers are generally made of plastic material or metal. They are rigid and have a certain volume such that the “cloud” of active principle projected in the inside remains in suspension up to the time of inhalation by the patient and is not deposited prematurely on the walls.

The volume of the inhalation chamber and its rigidity, necessary for its efficacy, therefore make this device unsuitable for everyday treatment, in particular in the business or school environment.

One solution for remedying this drawback is to offer an inhalation chamber, the walls of which can be compacted when not in use.

Chambers of this type known from the prior art have the drawback that they are only compatible with connectors of dosing aerosols or distribution means of a specific diameter, with a slight sealing play.

The chambers known from the prior art cannot be adapted to connectors of different sizes.

To remedy this drawback, the invention proposes an inhaling device of the type described above, characterised in that at least one end of the reservoir comprises an orifice for introduction and coupling of a connector of the dosing aerosol and/or a distribution means, of adjustable diameter to allow adaptation to the connectors of dosing aerosols and/or distribution means of different sizes.

According to other characteristics of the invention:
the introduction and coupling orifice is delimited by an annular collar of elastic material of diameter variable between two specific minimum and maximum diameters,
the introduction orifice is delimited by a collar with an inflatable annular chamber of diameter variable between two specific minimum and maximum diameters, the collar comprising on its outer wall at least one inflation valve communicating with the interior of its inflatable annular chamber,
the first mobile wall is a flexible wall,
the first mobile flexible wall is reinforced by a rigidification means mobile between a compact position and a deployed position,
the rigidification means comprises a second flexible wall lining the first flexible wall which with the first flexible wall delimits in a sealed fashion an inflatable cavity to allow the reservoir to occupy a reduced volume when the cavity is deflated and to allow the reservoir to be deployed in a rigid tubular form when the cavity is inflated, the first flexible wall comprising at least one cavity inflation valve,
the first mobile flexible wall is reinforced by a helical spring forming the rigidification means mobile between a spring compression position and a spring decompression position to allow the reservoir to occupy a reduced volume when the spring is compressed and to allow the reservoir to be deployed in a rigid tubular form when the spring is decompressed,
the first flexible wall is shaped as a bellows to allow the reservoir to occupy a reduced volume when the bellows is compressed and to allow the reservoir to be deployed into a substantially tubular rigid form when the bellows is decompressed,
the first mobile wall comprises a succession of concentric rigid tubular wall sections of increasing diameter forming a mobile telescopic wall to allow the reservoir to occupy a reduced volume when the sections are held coaxially in each other and to allow the reservoir to be deployed in the rigid tubular form when the sections are deployed axially,
the wall of the reservoir is made of a so-called memory elastomer material which is able to occupy each of the deployed and compact positions in a stable manner while passing through a plurality of unstable intermediate positions,
the mobile wall has a substantially tubular form and comprises at least two coaxial tubular sections of different diameters linked together, which are mobile between:
   a first relative position associated with a deployed position of the mobile wall in which the sections extend axially end to end,
   a second relative position associated with a compact position of the mobile wall in which at least one section is returned elastically and received in or on another section such that axially the wall only occupies the length of a single section,
the mobile wall comprises a succession of pairs, in the manner of odd and even, of coaxial tubular sections of decreasing diameter which are mobile between:
   a first relative position associated with a deployed position of the mobile wall in which all sections extend axially end to end,
   a second relative position associated with a compact position of the mobile wall in which each “even” section is returned elastically at least in or on an immediately consecutive “odd” section such that the wall only occupies the length of a single section,
a second opposite end of the reservoir comprises a neck fitted with a coupling opening allowing attachment to the treatment distribution means,
the neck is intended to receive in its introduction and coupling orifice an inspiration/expiration distributor comprising at least one valve for inspiration of the gas contained in the reservoir and one valve for expiration of the gas exhaled by the user, which distributor is interposed between the inhalation chamber and the distribution means,
the inspiration/expiration distributor is composed of a first and a second substantially tubular element joined together, the first tubular element being held in a complementary tubular portion of the distribution means, the second tubular element being held in the coupling orifice and receiving the inspiration valve on the inside, the expiration valve being interposed between the first and second elements,
the second end of the reservoir comprises a tubular coupling portion, the free end of which comprises the coupling orifice, an internal bore of which comprises at least one transverse shoulder face and which is intended to receive the second tubular element, a first annular section of the second element coming to rest on the free end face of the tubular coupling portion, a second tubular section of the second element being held in the internal bore of the tubular coupling portion resting on the transverse section face and a sealing collar of the second tubular section being held in a corresponding groove for the internal bore of the tubular coupling portion,
the intake valve is made of a flexible elastomer material and comprises a first truncated split section received at least partly in the second tubular section of the second element, and a second annular section clamped between a free end of the second tubular section of the second element and the transverse shoulder face of the internal bore of the tubular coupling portion,
the expiration valve is made of a flexible elastomer material and takes the form of a washer, an inner edge of which is clamped between the first annular section of the second element and the free face of the end of the tubular coupling portion, and an outer edge of which rests freely on a transverse shoulder face formed inside the first tubular element,
the first element comprises a first tubular section held in a complementary tubular section of the distribution means and a second truncated tubular section which comprises on the inside the transverse shoulder face,
the inspiration/expiration distributor is encased in the tubular neck of elastomer material formed in the second end of the reservoir,
the inspiration/expiration distributor comprises an external tubular element of elastomer material forming an expiration valve which is encased in the tubular neck of elastomer material formed in the second end of the reservoir, and an internal tubular element forming an inspiration valve which is encased coaxially in the external tubular element forming the expiration valve,
the neck of the second end of the reservoir comprises at least one first and one second neck section of first and second large inner diameters which are arranged on either side of a third section of the neck, known as a choke, of reduced inner diameter, and the external tubular element forming the expiration valve comprises at least a third and a fourth section of large outer diameters, respectively complementary to the first and second neck sections, arranged on each side of a fifth section of outer diameter complementary to the third neck section, to allow the external tubular element forming the expiration valve to be encased in the neck,
the external tubular element forming the expiration valve comprises at least a sixth and a seventh section of large inner diameter which are arranged on either side of an eighth section of reduced inner diameter forming an annular groove, and the internal tubular element forming the inspiration valve comprises at least a ninth section consisting of an annular collar encased in an eighth section of reduced inner diameter of the external tubular element to allow the internal tubular element forming the inspiration valve to be encased in the external tubular element forming the expiration valve,
the sixth, seventh and eighth sections of the external tubular element forming the expiration valve coincide axially respectively with the third, fourth and fifth sections of said external tubular element,
the first neck section is formed at a free end of the reservoir, the elastomer material of which it is made being of specific stiffness, and the third section corresponding to the external tubular element which is held in said first neck section comprises at least one piercing through the thickness of said third section, the elastomer material of the first section being mobile between a rest position in which it is applied to the outer surface of the third section and seals the piercing and an expiration position in which it is raised from the outer surface of the third section and releases the piercing when said third section is subjected internally to an expiration pressure,
the internal tubular element forming the inspiration valve comprises at least a tenth section which is attached to the ninth section forming the annular collar and which is turned towards the free end of the reservoir, said tenth section having a truncated form and comprising at its free end a transverse slot determining two lips which are able to move apart to allow passage of the inspiration gasses.

Other characteristics and advantages of the invention will appear from reading the detailed description below, for understanding of which reference is made to the attached drawings in which:

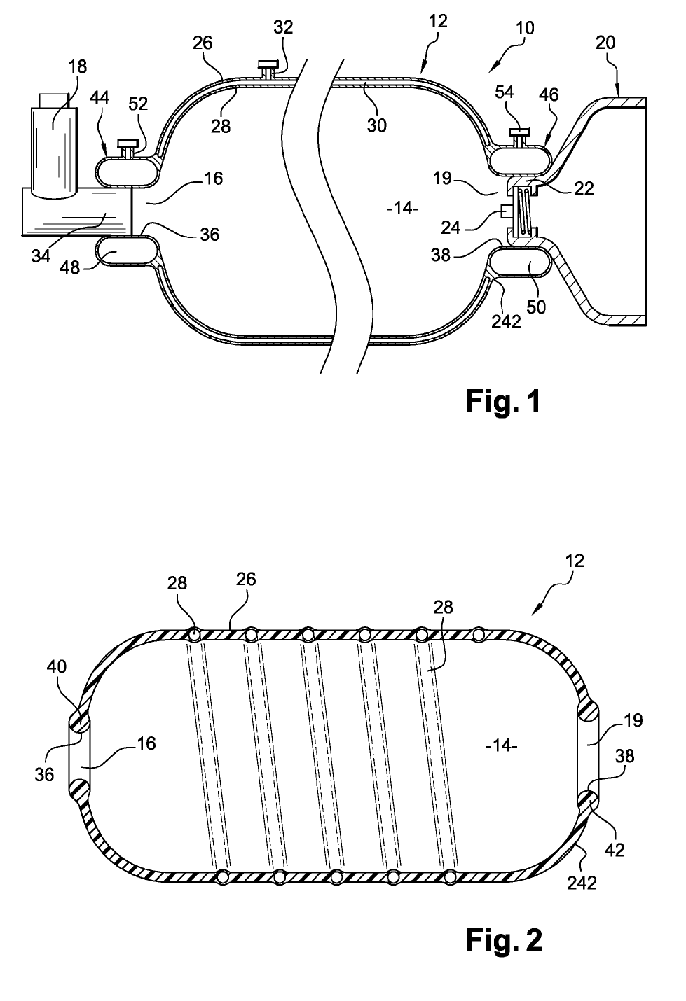

FIG. 1 shows a first embodiment of an inhaling device according to the invention;

FIG. 2 shows a second embodiment of an inhaling device according to the invention;

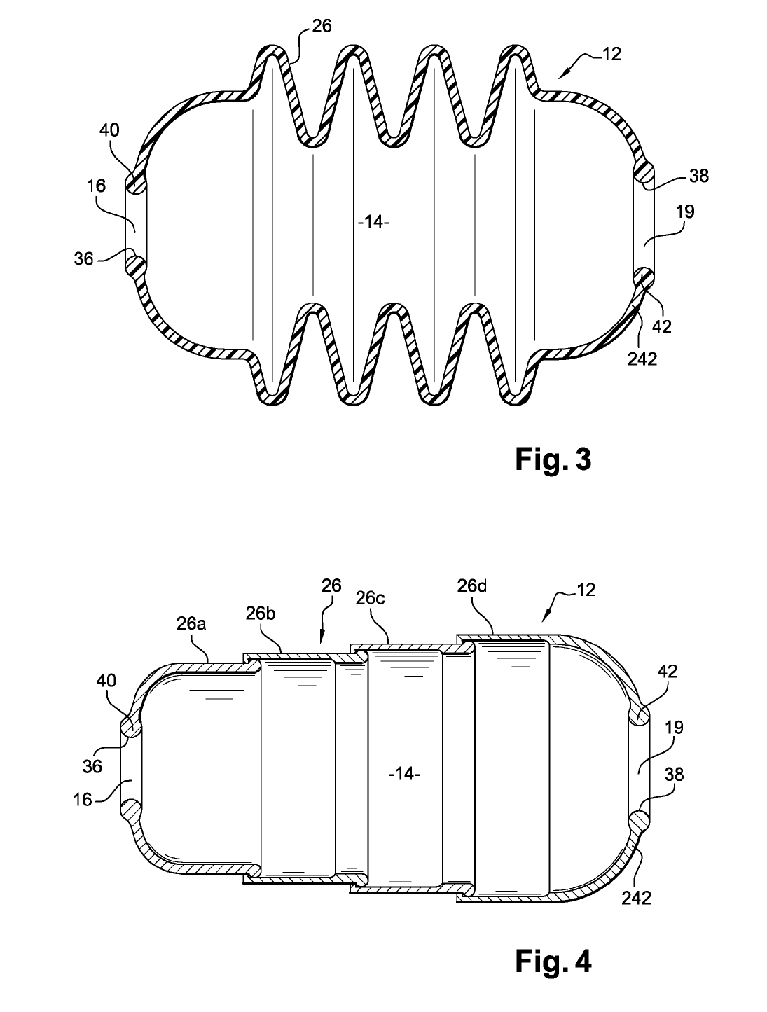

FIG. 3 shows a third embodiment of an inhaling device according to the invention;

FIG. 4 shows a fourth embodiment of an inhaling device according to the invention;

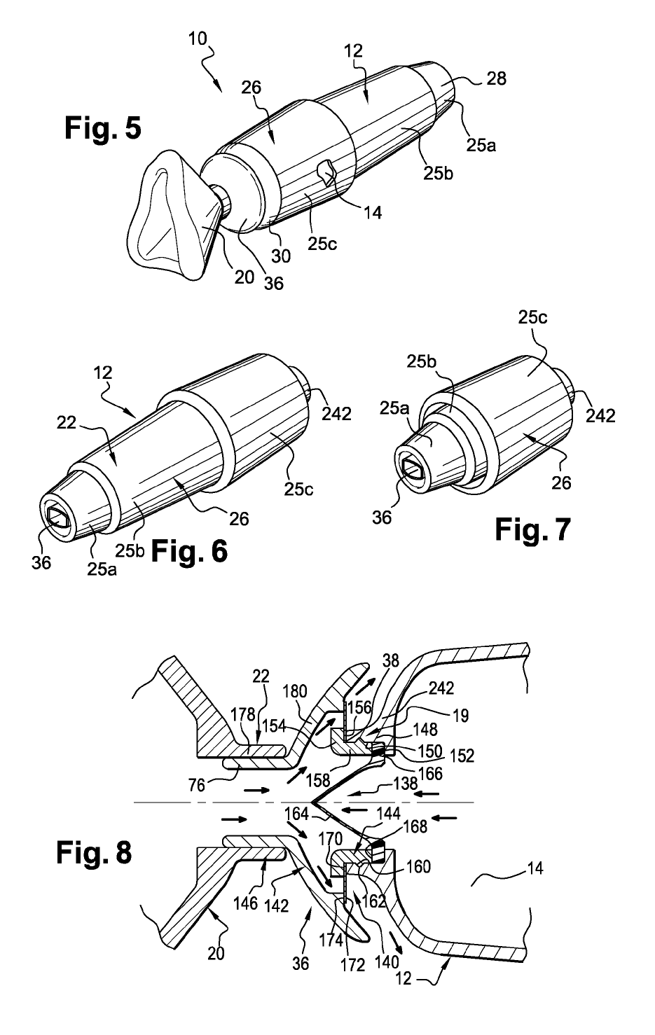

FIG. 5 is a perspective view of a fifth embodiment of an inhaling device according to the invention;

FIG. 6 is a perspective view of the single reservoir of the device in FIG. 5 in the deployed position of the mobile wall;

FIG. 7 is a perspective view of the single reservoir of the device in FIG. 5 in the compact position of the mobile wall;

FIG. 8 is a section view of the second end of the reservoir in FIG. 5 and a first embodiment of an inspiration/expiration distributor allowing its coupling to the treatment distribution means;

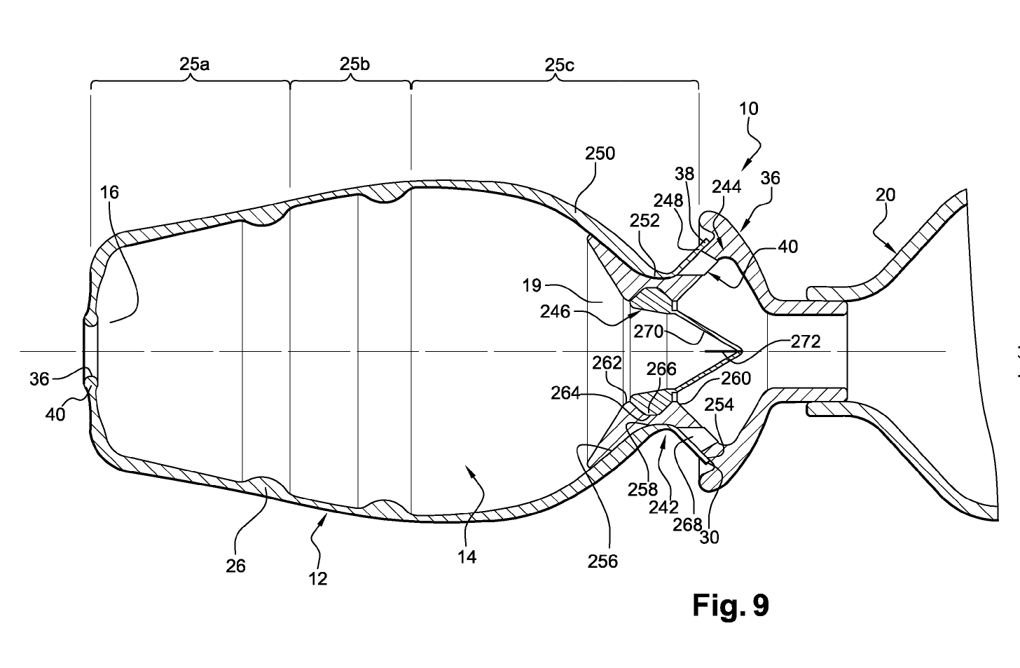

FIG. 9 is a section view of the second end of the reservoir in FIG. 5 and a second embodiment of an inspiration/expiration distributor allowing its coupling to the treatment distribution means.

In the description below, identical reference numerals designate identical parts or those with similar function.

FIGS. 1 to 5 show various embodiments of an inhaling device 10 produced in accordance with the invention.

In the known manner, such a device 10 is intended to allow the administration of inhaled treatments intended for treatment of bronchopulmonary disorders, asthma, bronchopathies or bronchiolitis.

In the known manner the device 10 comprises a substantially tubular axial reservoir 12 delimiting an inhalation chamber 14, a first end 16 of which is attached to a connector 34 of a dosing aerosol 18 containing an active principle and a second opposite end of which 19 is attached to a connector 22 of a treatment distribution means 20, in particular a mouth piece 22, a facial mask or a nasal distributor.

In the known manner the distribution means 20 comprises a main conduit forming the connection 22 inside which is arranged a valve 24 forming a non-return valve. The opening of the valve 24 is triggered by the reduced pressure caused by the inspiration of the user, and closure of this valve 24 is caused by the cessation of inspiration.

This configuration is not limitative for the invention and it will be understood that the valve 24 can, in all embodiments of the invention, be arranged in the second end 19 of the reservoir 12 without changing the nature of the invention.

In the known manner the reservoir 12 comprises at least one first wall 26 which is mobile between a compact position (not shown) and a deployed position depicted on the figures so as to allow said reservoir 12 to occupy a first position in which it occupies a reduced volume for transport and a second position in which it is deployed in an substantially rigid form for its use.

According to four embodiments shown on FIGS. 1 to 3, 5 to 7 and 9, the first mobile wall 26 is a flexible wall.

To allow the deployment or compacting of the reservoir 12, the first mobile flexible wall 26, as shown in the first and second embodiments of the invention, can be reinforced by a rigidification means 28 mobile between a compact position and a deployed position.

According to the first embodiment of the inhaling device shown in FIG. 1, the rigidification means comprises a second flexible wall 28 lining the first flexible wall 26, which with the first flexible wall 26 delimits in a sealed manner an inflatable cavity 30. The first flexible wall 26 comprises at least one valve 32 for inflating the cavity 30.

This configuration allows the reservoir 12 to occupy a reduced volume when the cavity 30 is deflated or conversely to be deployed in a rigid tubular form when the cavity 30 is inflated as shown in FIG. 1.

By simple inflation, the volume of which is low as it comprises inflating merely the cavity 30 between the double walls 26, 28, the chamber 12 becomes a perfectly operational inhalation chamber, the volume of which may become far greater than that of current small chambers.

Inflation can be performed by the patient himself or by any person in his environment.

According to a second embodiment of the inhaling device shown in FIG. 2, the first flexible mobile wall 26 is reinforced by a helical spring 28 forming the mobile rigidification means. This spring 28 is mobile between a compression position (not shown) of the spring 28 and a decompression position of the spring 28 shown in FIG. 2 to allow the reservoir 12 to occupy a reduced volume when the spring 28 is axially compressed and to allow the reservoir 12 to be deployed in a rigid tubular form when the spring 28 is decompressed.

The first flexible wall 26 may be glued directly to the spring 28. As a variant the wall 26 may be obtained by a moulding process and in this case the spring 28 will preferably be encased in the wall 26, the wall 26 then being moulded over the spring 28.

Also a means (not shown) for holding the wall 26 and spring 28 in compressed position may be provided such as a link or a strap allowing the reservoir 12 to be held in the compressed position to facilitate transport of the device 10.

According to a third embodiment of the inhaling device shown in FIG. 3, the first flexible wall 26 is shaped as a bellows to allow the reservoir 12 to occupy a reduced volume when the bellows is compressed axially and to allow the reservoir 12 to be deployed in a rigid substantially tubular form when the bellows is decompressed.

This third embodiment is based on an operating principle similar to the second embodiment with the difference that the first wall 26 is not reinforced. The simple shaping of the wall 26 obtained during production of said wall 26 by moulding suffices to give it the axial elasticity allowing its compression and decompression.

In a similar manner to the previous embodiment, a means (not shown) can then be provided for holding the wall 26 in compressed position such as link or a strap allowing the reservoir 12 to be held in compressed position in order to facilitate transport of the device 10.

According to a fourth embodiment of the inhaling device shown in FIG. 4, the first mobile wall 26 consists of a succession of concentric rigid tubular sections of wall 26a, 26b, 26c, 26d of increasing diameter forming a mobile telescopic wall 26. These sections 26a, 26b, 26c, 26d may be held coaxially inside each other in the compact position (not shown) to allow the reservoir to occupy a reduced volume, or deployed axially as shown in FIG. 4 to allow the reservoir 12 to be deployed in a rigid tubular form.

It will be understood that the first mobile wall 26 could comprise a greater or lesser number of sections without changing the nature of the invention.

According to a fifth embodiment of the inhaling device shown in FIGS. 5 to 7 and 9, the mobile wall 26 of the reservoir 12 is advantageously made of a so-called “memory” elastomer material which is able to occupy each of the deployed and compact positions in a stable manner while passing through a plurality of unstable intermediate positions.

More particularly, as shown in FIGS. 6 and 7, the mobile wall 26 has an substantially tubular form and comprises at least two coaxial tubular sections 25a, 25b, 25c of different diameters linked together.

In the preferred embodiment of the invention, the reservoir 12 is moulded integrally into the elastomer material. It comprises three coaxial sections 25a, 25b, 25c, the end sections 25a and 25c being intended to receive respectively the dosing aerosol and the distribution means 20.

The mobile wall 26 consists of coaxial sections 25a, 25b and 25c and is able to be moved manually between its deployed position in FIG. 6 and its compact position in FIG. 7.

To reach these positions the tubular coaxial sections 25a, 25b, 25c are mobile in relation to each other.

The coaxial sections 25a, 25b, 25c are thus mobile between:
a first relative position shown in FIG. 6 associated with the deployed position of the mobile wall 26 in which sections 25a, 25b and 25c extend axially end to end,
a second relative position shown in FIG. 7 associated with the compact position of the mobile wall 26 in which at least one section 25b is returned elastically and received in or on another section 25a, 25c such that the mobile wall 26 axially only occupies the length of the single section 25c.

Thus as shown in FIG. 7, which corresponds to a configuration of three sections 25a, 25b and 25c, section 25b is able to be returned elastically in the manner of a sock to be received in section 25c, and section 25b is simultaneously returned elastically in the manner of a sock onto section 25a such that the assembly of sections 25a, 25b and 25c no longer occupies more than the axial length of section 25c alone.

This configuration is not limited merely to a configuration comprising only three sections. In fact in the case of a plurality of sections, regarding as a first “odd” section an end section of the type of section 25a, the mobile wall could comprise a succession of pairs, in the manner of odd and even, of coaxial tubular sections of increasing or decreasing diameters which are then mobile between:
a first relative position associated with the deployed position of the mobile wall in which all sections extend axially end to end,
a second relative position associated with the compact position of the mobile wall in which each “even” section is returned at least on or in an immediately consecutive “odd” section such that the wall occupies only the length of a single section.

Thus, to simplify, one section out of every two is returned in or onto its adjacent section.

This configuration is particularly advantageous as it allows the creation of a reservoir 12 of great length by multiplying the number of sections.

It is noted that the reservoir 12 thus constituted is not only able to be compacted axially but also able to be compacted radially insofar as its constituent elastomer material can easily be crushed to allow for example storage of the reservoir 12 in a bag, a satchel or a coat pocket.

Several devices can be considered for ensuring the seal between the reservoir 12 and the dosing aerosol 18 and/or distribution means 20.

In general at least one end 16 or 19 of reservoir 12 comprises an orifice 36, 38 for coupling and introduction of a connector 34 of the dosing aerosol 16 and/or a connector 22 of the distribution means 20. The diameter of this orifice 36, 38 is adjustable to allows its adaptation to connectors 34, 22 of dosing aerosols and/or distribution means of different sizes.

The orifice 36, 38, as shown in FIGS. 2, 4 and 9, can be delimited by an annular collar 40, 42 of expanding elastic material according to a variable diameter between two determined minimum and maximum diameters.

As a variant as shown in FIG. 1, the introduction and coupling orifice 36, 38 can be delimited by a collar 44, 46 comprising an associated inflatable annular chamber 48, 50 of variable diameter adjustable between two determined minimum and maximum diameters. In this case the collar 44, 46 comprises on its outer wall at least one valve 52, 54 for inflation communicating with the interior of its inflatable annular chamber 48, 50. The inflation to a greater or lesser extent of the annular chamber 48, 50 allows the diameter of orifice 36, 38 to be increased or reduced as required as a function of the diameter of the connector 40, 22 to be introduced.

Also the second opposite end 19 of the reservoir 12 comprises a neck 242 with an introduction and coupling orifice 38 allowing connection to the treatment distribution means 20.

More precisely the reservoir 12 is not coupled directly to the treatment distribution means 20 but an inspiration/expiration distributor 36 is interposed between the reservoir 12 and the distribution means 20.

According to a first embodiment of the inspiration/expiration distributor 36 shown in FIG. 8, the coupling orifice 34 is intended to receive the inspiration/expiration distributor 36.

This distributor 36 interposed between the inhalation chamber and the distribution means comprises at least one valve 138 for inspiration of the gasses contained in the reservoir and a valve 140 for expiration of the gasses exhaled by the user.

In this first embodiment, the inspiration/expiration distributor 36 comprises a first substantially tubular element 142 and a second substantially tubular element 44 joined together.

The first tubular element 42 is held in a complementary tubular portion 146 of the connector 22 of the distribution means 20 and the second tubular element 44 is held in the orifice 38.

The second tubular element 44 also receives on the inside the inspiration valve 138.

Advantageously the expiration valve 140 is interposed between the first and second elements 142, 144.

More particularly the second end 19 of the reservoir 12 comprises a tubular coupling portion 148, the free end 156 of which comprises the introduction and coupling orifice 38, of which an internal bore 150 comprises at least one transverse shoulder face 152.

This tubular coupling portion 148 is thus intended to receive the second tubular element 144.

Thus a first annular section 154 of the second element 144 is held supported on the free end face 156 of the coupling tubular section 148, and a second tubular section 158 of the second element 144 is held in an internal bore 150 of the tubular coupling portion 148 resting on the transverse shoulder face 152.

The seal is achieved by means of a sealing collar 160 of the second tubular section 152 which is received in a corresponding groove 162 of the internal bore 150 of the coupling tubular section 148.

In this configuration the inspiration valve 138 is made of a flexible elastomer material and comprises a first split truncated section 164 held at least partly in the second tubular section 158 of the second element 154. The inspiration valve 138 also comprises, for its fixing, a second annular section 166 which is clamped between a free end 168 of the second tubular section 158 of the second element 144 and the transverse shoulder face 152 of the internal bore 150 of the tubular coupling portion 148.

According to a similar fixing method, the expiration valve 40 is made of a flexible elastomer material and has the form of a washer, an inner edge 170 of which is clamped between the first annular section 154 of the second element 144 and the free end face 156 of the tubular coupling portion 148, and the outer edge 172 of which rests freely on a transverse shoulder face 174 formed inside the first tubular element 142.

When a user of the distribution means 20 exhales, the expiration valve 140 is subject to pressure from the distribution means 20 and its outer edge 172 lifts from the shoulder face 174 allowing passage of the gasses.

Conversely when the user of the distribution means 20 inhales, the expiration valve 140 is subject to a reduced pressure from the distribution means 20 and its outer edge 172 seals against the shoulder face 174, preventing the passage of the gasses.

It is noted that to allow coupling of the first element 42 to the distribution means 20, the first element 142 comprises a first tubular section 176 which is held in a complementary tubular portion 178 of the connector 22 of the distribution means 20.

The first element 142 also comprises a second truncated tubular section 180 which on its inside comprises the transverse shoulder face 174.

According to a second embodiment of the inspiration/expiration distributor 36, to simplify the mounting of such a distributor 36 in the reservoir 12, the inspiration/expiration distributor 36 is encased in the tubular neck 242 of elastomer material formed in the second end 19 of the reservoir.

More particularly the inspiration/expiration distributor 36 comprises an external tubular element 244 of elastomer material forming the expiration valve which is encased in the tubular neck 242 of elastomer material formed in the second end 19 of the reservoir 12, and an internal tubular element 246 forming the inspiration valve which is encased coaxially in the tubular element 244 forming the expiration valve.

To allow the external tubular element 244 to be encased in the reservoir 12, the neck 242 of the second end 19 of the reservoir 12 comprises at least one first and one second neck section 248, 250 of large first and second inner diameter which are arranged on either side of a third neck section 252 known as a choke of reduced inner diameter.

The external tubular element 244 forming the expiration valve comprises at least a third and a fourth section 254, 256 of large outer diameter, respectively complementary to the first and second neck sections 248 and 250, which are arranged on either side of a fifth section 258 of outer diameter complementary to the third neck section 252 to allow the external tubular element 244 forming the expiration valve to be encased in the neck 242.

Naturally the inspiration/expiration distributor 36 is encased by deforming suitably the neck 242 of elastomer material on its introduction into said neck 242.

Also the external tubular element 244 forming the expiration valve comprises at least a sixth and a seventh section 260, 262 of large inner diameter arranged on either side of an eighth section 264 of reduced inner diameter forming an annular groove. The internal tubular element 246 forming the inspiration valve comprises at least a ninth section 266 consisting of an annular collar which is encased in the eighth section 264 of reduced inner diameter of the external tubular element 244 to allow the internal tubular element 246 forming the inspiration valve to be encased in the external tubular element 244 forming the expiration valve.

Advantageously to reduce the axial size of the distributor 36 it is desirable for the expiration valve 244 and the inspiration valve 246 to coincide axially.

To this end the sixth, seventh and eighth sections 260, 262, 264 of the external tubular element 244 forming the expiration valve coincide axially respectively with the third, fourth and fifth sections 254, 256, 258 of said external tubular element 244.

This configuration is not limitative of the invention and the sixth, seventh and eighth sections 260, 262, 264 of the external tubular element 244 forming the expiration valve can be offset axially in relation to the third, fourth and fifth sections 254, 256, 258 of said external tubular element 244.

In order to constitute the expiration valve 36 as shown in the single figure, the first neck section 248 is formed at a free end 19 of the reservoir 12 and its constituent elastomer material has a specific stiffness.

Also the third section corresponding section 254 of the external tubular element 244 which is held in said first neck section 248 comprises at least one piercing 268 through the thickness of said third section 254.

In this way the elastomer material of the first section is mobile between a rest position shown in the single figure, in which it is applied to the outer surface of the third section 248 and seals the piercing 268, and an expiration position (not shown) in which it is lifted from the outer surface of the third section 248 and releases the piercing 268 when said third section 248 is subject internally to expiration pressure.

Thus a distributor 36 is produced in a very simple manner.

Finally the internal tubular element 246 forming the inspiration valve comprises at least a tenth section 270 which is attached to the ninth section 266 forming the annular collar and which is turned towards the free end 19 of reservoir 12 and which has a truncated form. This tenth section 270 comprises at its free end a transverse slot 272 determining two lips which are able to move apart to allow passage of the inspiration gasses.

It will therefore be understood that to achieve the mounting of distributor 36 in the reservoir 12, first the internal element 246 forming the inspiration valve is inserted in the external element 244 forming the expiration valve and then the resulting assembly is inserted in the neck 242 of reservoir 12.

The invention thus proposes a device 10 which, when occupying its compact position, can be reduced to a very small volume and thus held in a pocket, a handbag, a satchel or similar. Chamber 12 of device 10 in this state is much less bulky and voluminous than the current inhalation chambers intended for everyday treatment. The device 10 can be assembled very easily.

The invention therefore proposes an inhaling device 10 which can easily be transported, which is particularly practical and simple to use.

1.-26. (canceled)

27. Inhaling device (10), in particular for administration of inhaled treatments intended to treat bronchopulmonary disorders, comprising an axial substantially tubular reservoir (12) delimiting an inhalation chamber (14), a first end (16) of which is attached to a dosing aerosol (18) containing an active principle and a second opposite end (19) of which is attached to a treatment distribution means (20), in particular a mouth piece or a facial mask, the reservoir (12) comprising at least one first wall (26) mobile between a compact position and deployed position so as to allow said reservoir (12) to occupy a first position in which it occupies a reduced volume for transport, and a second position in which it is deployed in a substantially rigid form for use, at least one end (16, 19) of the reservoir (12) comprising an orifice (36, 38) for introduction and coupling of a connector (34, 22) of the dosing aerosol (18) and/or the distribution means (20) of adjustable diameter, characterised in that said second opposite end (19) of the reservoir (12) comprises a neck (242) with an introduction and coupling orifice (38) and intended to receive in its introduction and coupling orifice (38) an inspiration/expiration distributor (36) comprising at least one valve (138, 244) for inspiration of the gasses contained in the reservoir and one valve (140, 246) for expiration of the gasses exhaled by the user, which is interposed between the inhalation chamber (14) and the distribution means (20).

28. Inhaling device (10) according to claim 27, characterised in that the introduction and coupling orifice (36, 38) is delimited by an annular collar (40, 42) of elastic material of a diameter variable between two determined minimum and maximum diameters, or by a collar (44, 46) comprising an inflatable annular chamber (48, 50) of a diameter variable between two determined minimum and maximum diameters, the collar (44, 46) comprising on its outer wall at least one inflation valve (52, 54) communicating with the interior of its inflatable annular chamber (48, 50).

29. Inhaling device (10) according to claim 27, characterised in that the first mobile wall (26) is a flexible wall (26).

30. Inhaling device (10) according to claim 29, characterised in that the first mobile flexible wall (26) is reinforced by a rigidification means (28) mobile between a compact position and a deployed position.

31. Inhaling device (10) according to claim 30, characterised in that the rigidification means comprises a second flexible wall (28) lining the first flexible wall (26) which with the first flexible wall (26) delimits in a sealed manner an inflatable cavity (30) to allow the reservoir (12) to occupy a reduced volume when the cavity (30) is deflated and to allow the reservoir (12) to be deployed in a rigid tubular form when the cavity (30) is inflated, the first flexible wall (26) comprising at least one valve (32) for inflating the cavity.

32. Inhaling device (10) according to claim 29, characterised in that the first flexible wall (26) is shaped as a bellows to allow the reservoir (12) to occupy a reduced volume when the bellows is compressed and to allow the reservoir (12) to be deployed in a substantially tubular rigid form when the bellows is decompressed.

33. Inhaling device (10) according to claim 27, characterised in that the first mobile wall (26) comprises a succession of concentric rigid tubular wall sections (26a, 26b, 26c, 26d) of increasing diameter forming a telescopic mobile wall (26) which allows the reservoir (12) to occupy a reduced volume when sections (26a, 26b, 26c, 26d) are received coaxially into each other and to allow the reservoir (12) to be deployed in a rigid tubular form when the sections (26a, 26b, 26c, 26d) are deployed axially.

34. Inhaling device (10) according to claim 29, characterised in that the wall (26) of reservoir (12) of substantially tubular form is made of a so-called memory elastomer material and comprises at least two tubular coaxial sections (25a, 25b, 25c) of different diameters linked together which are mobile between:
a first relative position associated with the deployed position of the mobile wall (26) in which the sections (25a, 25b, 25c) extend axially end to end,
a second relative position associated with the compact position of the mobile wall (26) in which at least one section (25b) is returned elastically and received in or on another section (25a, 25c) such that the wall (26) axially only occupies the length of a single section (25c).

35. Device (10) according to claim 34, characterised in that the inspiration/expiration distributor (36) is composed of a first (142) and a second substantially tubular element (144) joined together, the first tubular element (142) being held in a complementary tubular portion (146) of the distribution means (20), the second tubular element (144) being held in the coupling orifice (38) and receiving on the inside the inspiration valve (138), and the expiration valve (140) is interposed between the first and second elements (142, 144).

36. Device (10) according to claim 35, characterised in that the second end (19) of the reservoir (12) comprises a tubular coupling portion (148) of which the free end (56) comprises the coupling orifice (38), of which an internal bore (150) comprises at least one transverse shoulder face (152) and which is intended to receive the second tubular element (144), a first annular section (154) of the second element (144) being held resting on the free end face (156) of the tubular coupling portion (148), a second tubular section (158) of the second element (144) being held in the internal bore (150) of the tubular coupling portion (148) resting on the transverse shoulder face (152) and a sealing collar (160) of the second tubular section (158) being held in a corresponding groove (162) of the internal bore (150) of the tubular coupling portion (148).

37. Device (10) according to claim 36, characterised in that the inspiration valve (138) is made of a flexible elastomer material and comprises a first truncated split section (164) which is held at least partly in the second tubular section (158) of the second element (144), and a second annular section (166) which is clamped between a free end (168) of the second tubular section (158) of the second element (144) and the transverse shoulder face (152) of the internal bore (150) of the tubular coupling portion (148).

38. Device (10) according to claim 36, characterised in that the expiration valve (40) is made of a flexible elastomer material and takes the form of a washer, an inner edge (172) of which is clamped between the first annular section (154) of the second element (144) and the free end face (156) of the tubular coupling portion (148), and the outer edge (172) of which rests freely on a transverse shoulder face (174) formed inside the first tubular element (142).

39. Device (10) according to claim 38, characterised in that the first element (142) comprises a first tubular section (176) which is held in a complementary tubular section (178) of the distribution means (20) and a second truncated tubular section (180) which comprises on the inside the transverse shoulder face (174).

40. Device (10) according to claim 39, characterised in that the first element (142) comprises a first tubular section (176) which is received in a complementary tubular portion (178) of the distribution means (20) and a second truncated tubular section (180) which on the inside comprises the transverse shoulder face (174).

41. Inhaling device (10) according to claim 27, characterised in that the inspiration/expiration distributor (36) comprises an external tubular element (244) of elastomer material forming said expiration valve encased in the tubular neck (242) of elastomer material formed in the second end (19) of the reservoir (12), and an internal tubular element (246) forming said inspiration valve encased coaxially in the external tubular element (244) forming the expiration valve.

42. Device (10) according to claim 41, characterised in that the neck (242) of the second end of the reservoir comprises at least a first and a second neck section (248, 250) of large first and second inner diameters which are arranged on either side of a third neck section (252) called a choke of reduced inner diameter, and in that the external tubular element (244) forming the expiration valve comprises at least a third and a fourth section (254, 256) of large outer diameters, respectively complementary to the first and second neck sections (248, 250), arranged on either side of a fifth section (258) of outer diameter complementary to the third neck section (252) to allow the external tubular element (244) forming the expiration valve to be encased in the neck (242).

43. Device (10) according to claim 41, characterised in that the external tubular element (242) forming the expiration valve comprises at least a sixth and a seventh section (260, 262) of large inner diameters which are arranged on either side of an eighth section (264) of reduced inner diameter forming an annular groove, and in that the internal tubular element (246) forming the inspiration valve comprises at least a ninth section (266) comprising an annular collar which is encased in the eighth section (264) of reduced inner diameter of the external tubular element (244) to allow the internal tubular element (246) forming the inspiration valve to be encased in the external tubular element (244) forming the expiration valve.

44. Device (10) according to claim 42, characterised in that the first neck section (248) is formed at a free end (19) of the reservoir (12) and in that its constituent elastomer material is of a specific stiffness, and in that the corresponding third section (254) of the external tubular element (244) which is held in said first neck section (248) comprises at least one piercing (268) through the thickness of said third section (254), the elastomer material of the first section (248) being mobile between a rest position in which it is applied to the outer surface of the third section (254) and seals the piercing (268) and an expiration position in which it is raised from the outer surface of the third section (254) and releases the piercing (268) when said third section (254) is subject internally to an expiration pressure.
